(19) United States
(12) Patent Application Publication
(10) Pub. No.: US 2022/0185791 A1
(43) Pub. Date: Jun. 16, 2022

(54) POLYMORPHS OF A CARDIAC TROPONIN ACTIVATOR

(71) Applicants: Amgen Inc., Thousand Oaks, CA (US); Cytokinetics,Inc., South San Francisco, CA (US)

(72) Inventors: Stephanie AZALI, Thousand Oaks, CA (US); Mary CHAVES, Thousand Oaks, CA (US); Justin MALINOWSKI, Thousand Oaks, CA (US); Steven MENNEN, Thousand Oaks, CA (US); Darren REID, Thousand Oaks, CA (US)

(21) Appl. No.: 17/593,113

(22) PCT Filed: Mar. 12, 2020
(86) PCT No.: PCT/US2020/022221

§ 371 Date: Sep. 9, 2021

Related U.S. Application Data
(60) Provisional application No. 62/817,194, filed on Mar. 12, 2019.

(51) Int. Cl. C07D 401/06

(52) U.S. Cl. CPC C07D 401/06; C07B 2200/13

(57) ABSTRACT

Provided herein are free base crystalline forms, crystalline salts, and solvates of Compound B.

### BACKGROUND

The compound (1R,3R,5R)-N-((R)-(4-chloro-2,5-difluorophenyl)(cyclopropyl)methyl)-2-(5-(methylsulfonyl)nicotinoyl)-2-azabicyclo[3.1.0]hexane-3-carboxamide, is useful as a cardiac troponin activator:

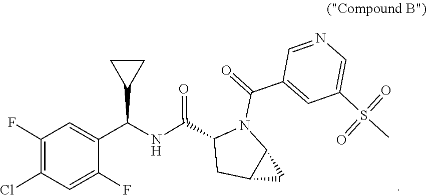 [SMILES: CS(=O)(=O)c1cncc(C(=O)N2[C@@H](C(=O)N[C@@H](c3cc(F)c(Cl)cc3F)C3CC3)C[C@H]3C[C@H]32)c1]

There is a need for various new salt and crystalline forms of Compound B with different chemical and physical stabilities, and formulations and uses of the same.

### SUMMARY

Provided herein are crystalline forms of Compound B or a salt thereof, including free base crystalline forms, crystalline salts, and crystalline solvates. In some embodiments, provided herein is the free base anhydrous crystalline Form I of Compound B. In some embodiments, provided herein is the free base monohydrate crystalline Form II of Compound B. In some embodiments, provided herein is the crystalline form of Compound B hydrochloride salt. In some embodiments, provided herein is the crystalline form of Compound B and acetonitrile. In some embodiments, provided herein is the crystalline form of Compound B and dichloroethane. In some embodiments, provided herein is the crystalline form Compound B and nitromethane.

Also provided are pharmaceutical compositions comprising the crystalline form of Compound B or salt thereof disclosed herein and a pharmaceutical acceptable carrier.

Further provided are methods of treating heart failure in a subject in need thereof comprising administering to the subject the crystalline form of Compound B or salt thereof disclosed herein in an amount effect to treat heart failure.

### BRIEF DESCRIPTION OF FIGURES

FIG. 1 depicts an X-ray powder diffraction (“XRPD”) pattern of the free base anhydrous crystalline Form I.

FIG. 2 depicts a differential scanning calorimetry (“DSC”) thermograph of the free base anhydrous crystalline Form I.

FIG. 3 depicts a thermogravimetric analysis (“TGA”) trace of the free base anhydrous crystalline Form I.

FIG. 4 depicts a dynamic vapor sorption (“DVS”) graph of the free base anhydrous crystalline Form I.

FIG. 5 depicts an X-ray powder diffraction (“XRPD”) pattern of the free base monohydrate crystalline Form II.

FIG. 6 depicts a differential scanning calorimetry (“DSC”) thermograph of the free base monohydrate crystalline Form II.

FIG. 7 depicts a thermogravimetric analysis (“TGA”) trace of the free base monohydrate crystalline Form II.

FIG. 8 depicts a dynamic vapor sorption (“DVS”) graph of the free base monohydrate crystalline Form II.

FIG. 9 depicts an overlay of the XRPD patterns of free base crystalline Form I (top) and Form II (bottom).

FIG. 10 depicts an X-ray powder diffraction (“XRPD”) pattern of the crystalline hydrochloride salt.

FIG. 11 depicts a differential scanning calorimetry (“DSC”) thermograph of the crystalline hydrochloride salt.

FIG. 12 depicts a thermogravimetric analysis (“TGA”) trace of the crystalline hydrochloride salt.

FIG. 13 depicts an X-ray powder diffraction (“XRPD”) pattern of the acetonitrile solvate.

FIG. 14 depicts a differential scanning calorimetry (“DSC”) thermograph of the acetonitrile solvate.

FIG. 15 depicts a thermogravimetric analysis (“TGA”) trace of the acetonitrile solvate.

FIG. 16 depicts an X-ray powder diffraction (“XRPD”) pattern of the dichloroethane solvate.

FIG. 17 depicts a differential scanning calorimetry (“DSC”) thermograph of the dichloroethane solvate.

FIG. 18 depicts a thermogravimetric analysis (“TGA”) trace of the dichloroethane solvate.

FIG. 19 depicts an X-ray powder diffraction (“XRPD”) pattern of the nitromethane solvate.

FIG. 20 depicts a differential scanning calorimetry (“DSC”) thermograph of the nitromethane solvate.

FIG. 21 depicts a thermogravimetric analysis (“TGA”) trace of the nitromethane solvate.

### DETAILED DESCRIPTION

The present disclosure provides various forms of (1R,3R,5R)-N-((R)-(4-chloro-2,5-difluorophenyl)(cyclopropyl)methyl)-2-(5-(methylsulfonyl)nicotinoyl)-2-azabicyclo[3.1.0]hexane-3-carboxamide, termed “Compound B” herein, and having a structure of:

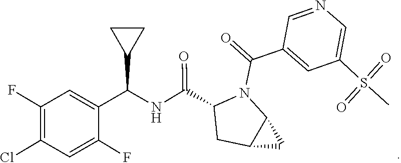 [SMILES: CS(=O)(=O)c1cncc(C(=O)N2[C@@H](C(=O)N[C@@H](c3cc(F)c(Cl)cc3F)C3CC3)C[C@H]3C[C@H]32)c1]

Embodiments of free base forms, salt forms, and solvates of Compound B can be characterized by one or more of the parameters described in further detail below.

### Free Base Crystalline Forms of Compound B

Provided herein are free base crystalline forms of Compound B. In embodiments, the free base crystalline forms of Compound B can be nonionic forms of Compound B. In embodiments, the free base crystalline forms of Compound B can be anhydrous. In embodiments, the free base crystalline forms of Compound B can be a monohydrate.

### Free Base Anhydrous Crystalline Form I

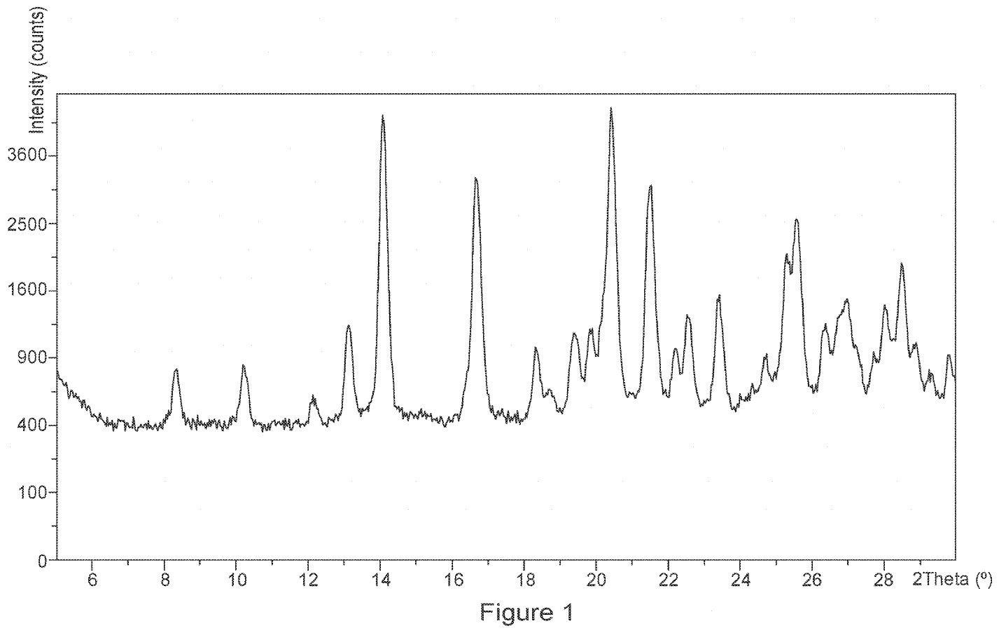

Free base anhydrous crystalline form I of Compound B (“Form I”) can be characterized by an X-ray powder diffraction pattern, obtained as set forth in the Examples, having peaks at about 8.31, 10.20, 13.11, 14.07, and 16.65±0.2° 2θ using Cu Kα radiation. Form I optionally can be further characterized by an X-ray powder diffraction pattern having additional peaks at about 20.42, 21.49, 22.57, 23.39, 25.27, and 25.60±0.2° 2θ using Cu Kα radiation. Form I optionally can be further characterized by an X-ray powder diffraction pattern having additional peaks at about 18.34, 19.36, 19.84, 22.21, 24.70, 26.31, 26.97, 28.02, 28.49, and 28.91±0.2° 2θ using Cu Kα radiation. Form I optionally can be characterized by an X-ray powder diffraction pattern having peaks shown in Table 1 set forth in the Examples. In some embodiments, Form I has an X-ray powder diffraction pattern substantially as shown in FIG. 1, wherein by “substantially” is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

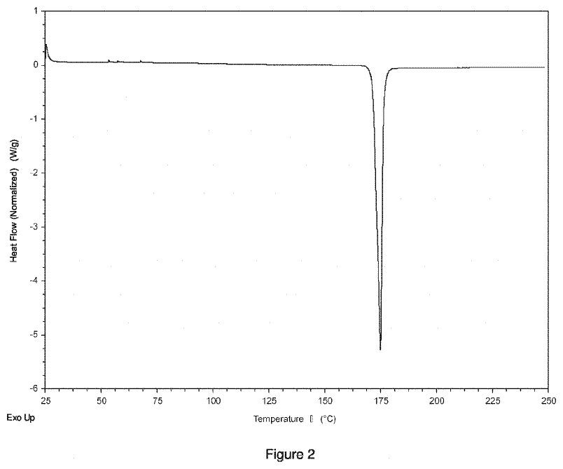

Differential scanning calorimetry (DSC) thermographs were obtained, as set forth in the Examples, for Form I. The DSC curve indicates an endothermic transition at about 175° C.±3° C. Thus, in some embodiments, Form I can be characterized by a DSC thermograph having a decomposition endotherm with an onset in a range of about 170° C. to about 180° C. For example, in some embodiments Form I is characterized by DSC, as shown in FIG. 2.

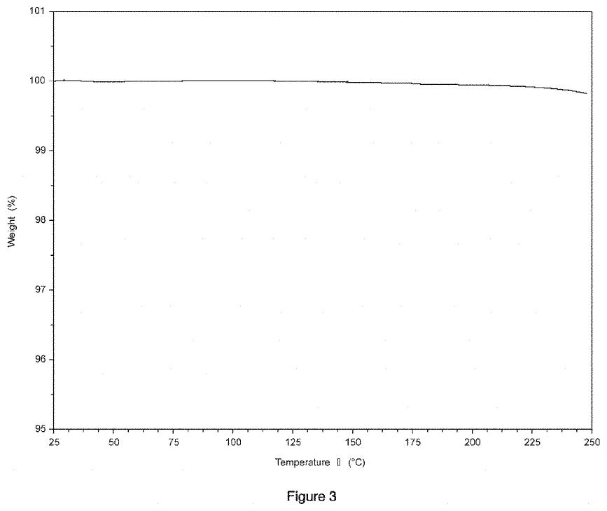

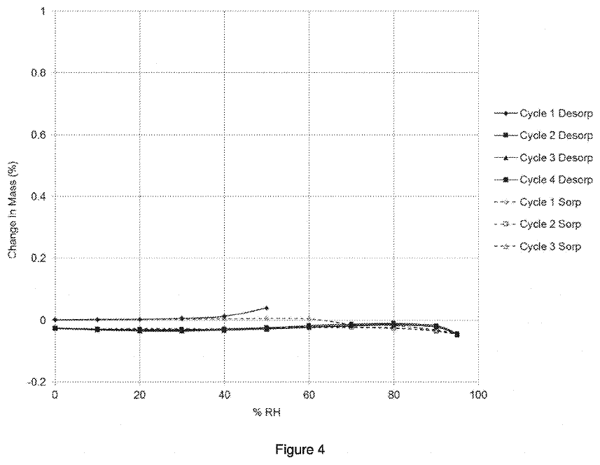

Form I also can be characterized by thermogravimetric analysis (TGA). Thus, Form I can be characterized by a weight loss in a range of about 0% to about 0.5% with an onset temperature in a range of about 25° C. to about 35° C. For example, Form I can be characterized by a weight loss of about 0.05%, up to about 200° C. In some embodiments, Form I has a thermogravimetric analysis substantially as depicted in FIG. 3, wherein by “substantially” is meant that the reported TGA features can vary by about ±5° C. In embodiments, Form I has a dynamic vapor sorption (“DVS”) substantially as shown in FIG. 4.

### Free Base Monohydrate Crystalline Form II

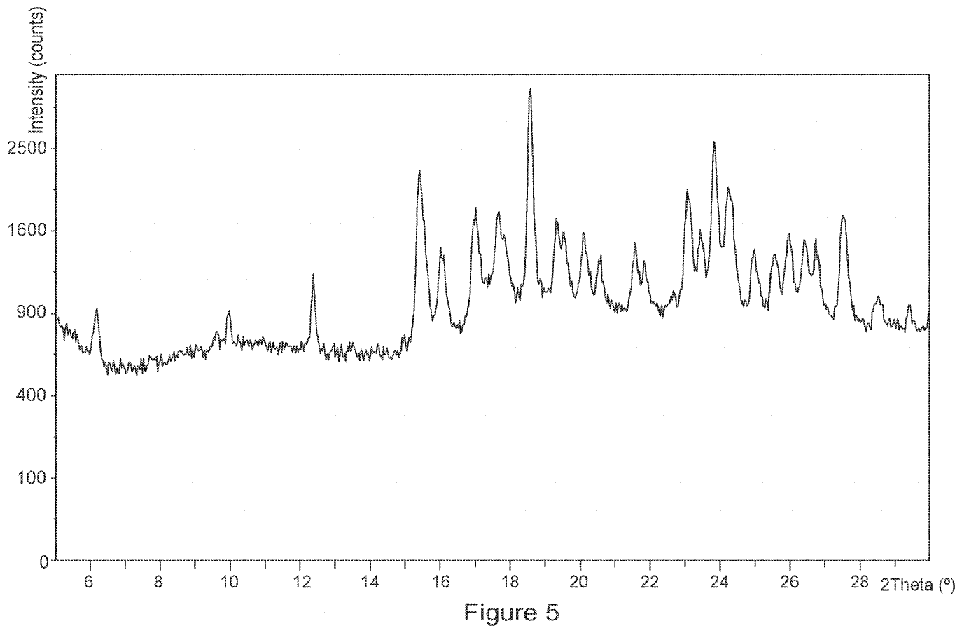

Free base monohydrate crystalline form II of Compound B (“Form II”) can be characterized by an X-ray powder diffraction pattern, obtained as set forth in the Examples, having peaks at about 6.19, 9.96, 12.37, 15.40, and 16.04±0.2° 2θ using Cu Kα radiation. Form II optionally can be further characterized by an X-ray powder diffraction pattern having additional peaks at about 16.97, 17.65, 18.57, 19.32, 20.10, 21.56, 23.08, 23.44, 23.83, 24.22, and 27.51±0.2° 2θ using Cu Kα radiation. Form II optionally can be further characterized by an X-ray powder diffraction pattern having additional peaks at about 20.54, 24.95, 25.51, 26.76, 28.49, and 29.43±0.2° 2θ using Cu Kα radiation. Form II optionally can be characterized by an X-ray powder diffraction pattern having peaks shown in Table 2 set forth in the Examples. In some embodiments, Form II has an X-ray powder diffraction pattern substantially as shown in FIG. 5, wherein by “substantially” is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

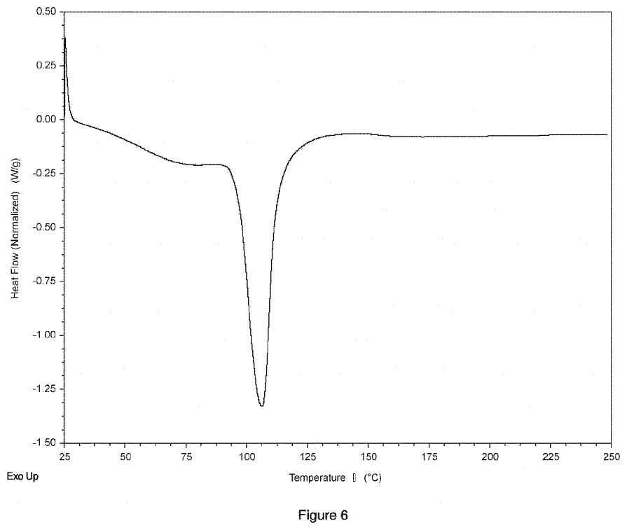

Differential scanning calorimetry (DSC) thermographs were obtained, as set forth in the Examples, for Form II. The DSC curve indicates an endothermic transition at about 106° C.±3° C. Thus, in some embodiments, Form II can be characterized by a DSC thermograph having a decomposition endotherm with an onset in a range of about 100° C. to about 115° C. For example, in some embodiments Form II is characterized by DSC, as shown in FIG. 6.

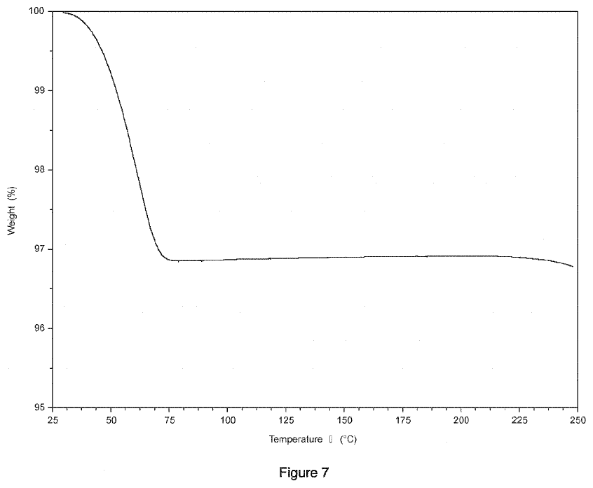

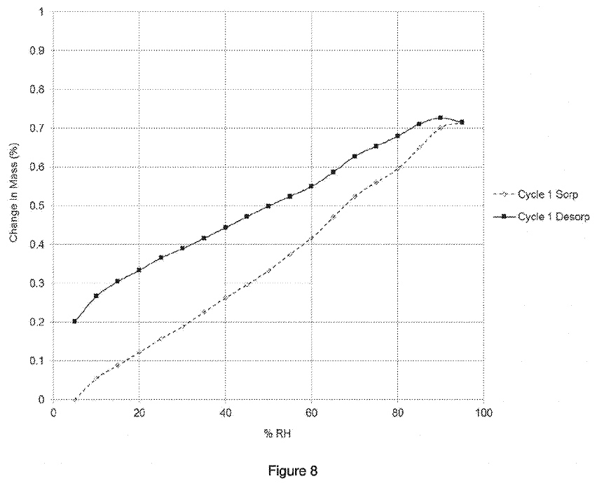

Form II also can be characterized by thermogravimetric analysis (TGA). Thus, Form II can be characterized by a weight loss in a range of about 2.6% to about 4.6% with an onset temperature in a range of about 30° C. to about 50° C. For example, Form II can be characterized by a weight loss of about 3.6%, up to about 100° C. In some embodiments, Form II has a thermogravimetric analysis substantially as depicted in FIG. 7, wherein by “substantially” is meant that the reported TGA features can vary by about ±5° C. In embodiments, Form II has a dynamic vapor sorption (“DVS”) substantially as shown in FIG. 8.

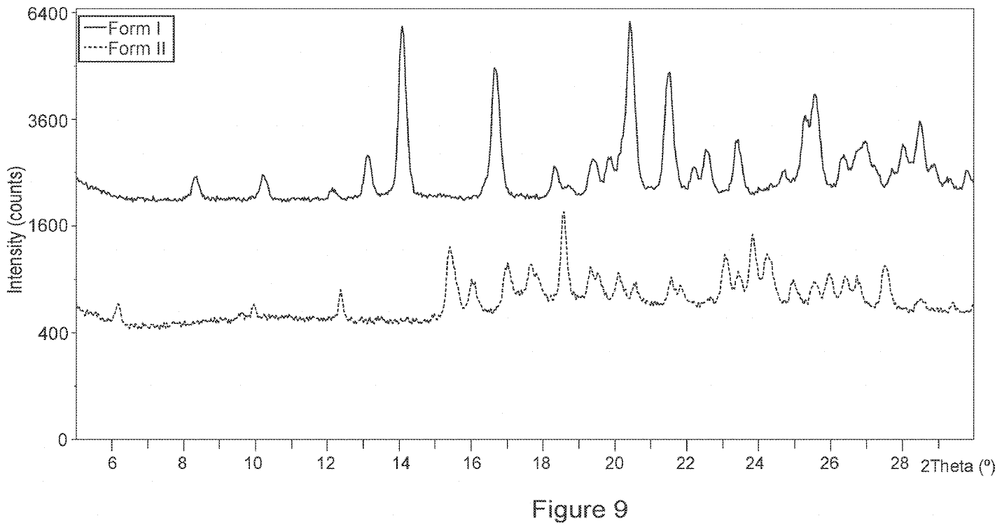

A summary of the distinct XRPD peaks in free base crystalline forms I and II can be seen in Table 3 and an overlay of the two different crystalline forms is shown in FIG. 9.

### Compound B Salts

### Crystalline Hydrochloride Salt

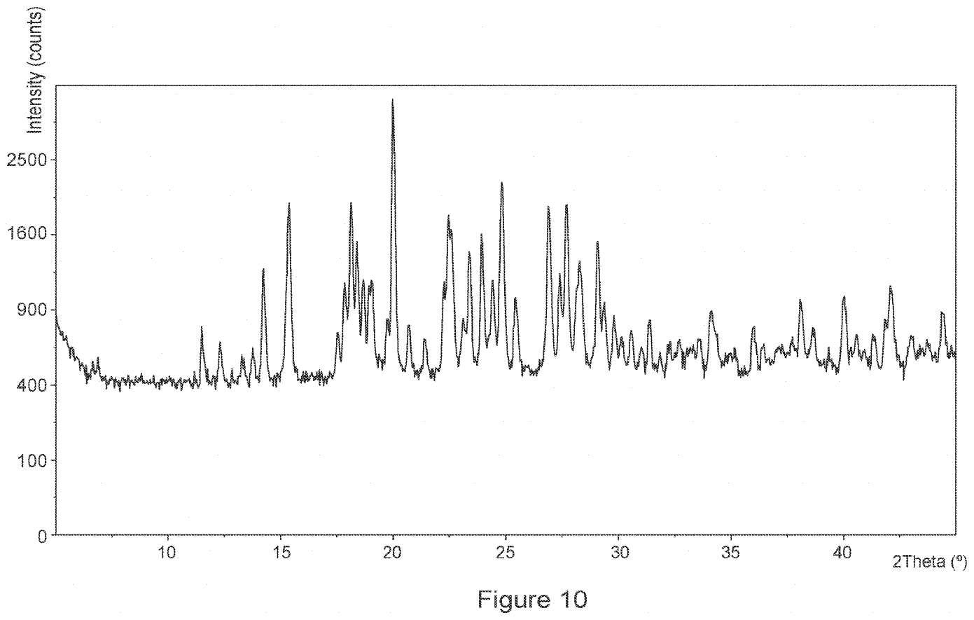

Crystalline form of Compound B hydrochloride salt (“hydrochloride salt”) can be characterized by an X-ray powder diffraction pattern, obtained as set forth in the Examples, having peaks at about 15.37, 18.13, 20.00, 22.45, 24.84, 26.91, and 27.71±0.2° 2θ using Cu Kα radiation. The hydrochloride salt optionally can be further characterized by an X-ray powder diffraction pattern having additional peaks at about 14.23, 17.83, 18.40, 18.68, 18.94, 19.07, 22.23, 22.45, 22.62, 23.39, 23.94, 24.42, 25.42, 27.39, 28.31, 29.08, 40.01, and 42.09±0.2° 2θ using Cu Kα radiation. The hydrochloride salt optionally can be further characterized by an X-ray powder diffraction pattern having additional peaks at about 11.50, 17.54, 19.73, 20.71, 23.09, 29.38, 29.80, 31.38, 34.09, 38.09, 44.39±0.2° 2θ using Cu Kα radiation. The hydrochloride salt optionally can be characterized by an X-ray powder diffraction pattern having peaks shown in Table 4 set forth in the Examples. In some embodiments, the hydrochloride salt has an X-ray powder diffraction pattern substantially as shown in FIG. 10, wherein by “substantially” is meant that the reported peaks can vary by about ±0.2°.

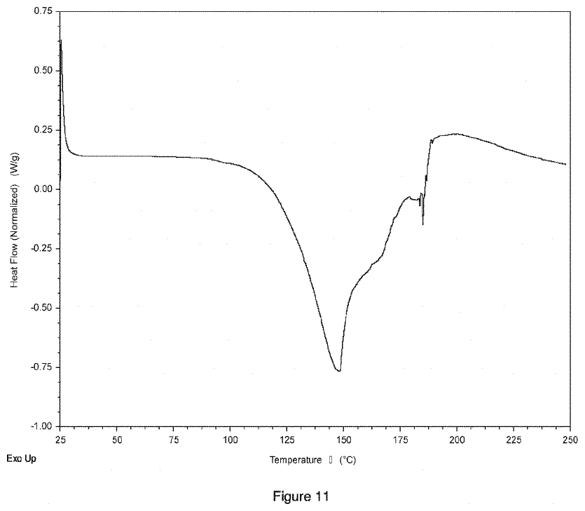

Differential scanning calorimetry (DSC) thermographs were obtained, as set forth in the Examples, for the hydrochloride salt. The DSC curve indicates an endothermic transition at about 148° C.±3° C. Thus, in some embodiments, the hydrochloride salt can be characterized by a DSC thermograph having a decomposition endotherm with an onset in a range of about 140° C. to about 155° C. For example, in some embodiments the hydrochloride salt is characterized by DSC, as shown in FIG. 11.

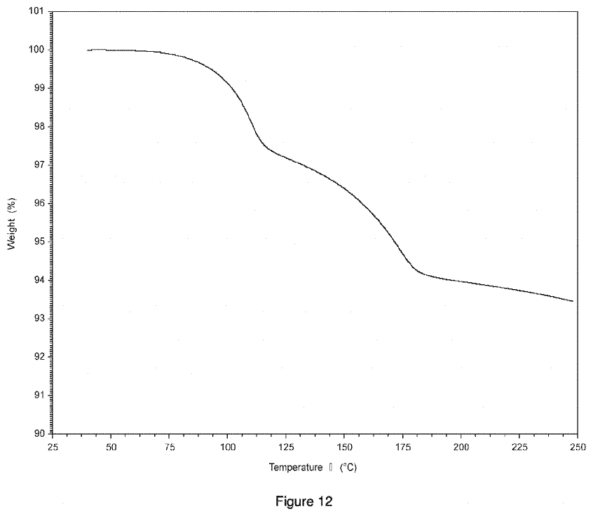

The hydrochloride salt also can be characterized by thermogravimetric analysis (TGA). Thus, the hydrochloride salt can be characterized by a weight loss in a range of about 5% to about 7% with an onset temperature in a range of about 70° C. to about 90° C. For example, the hydrochloride salt can be characterized by a weight loss of about 6.0%, up to about 200° C. In some embodiments, the hydrochloride salt has a thermogravimetric analysis substantially as depicted in FIG. 12, wherein by “substantially” is meant that the reported TGA features can vary by about ±5° C.

### Compound B Solvates

### Acetonitrile Solvate

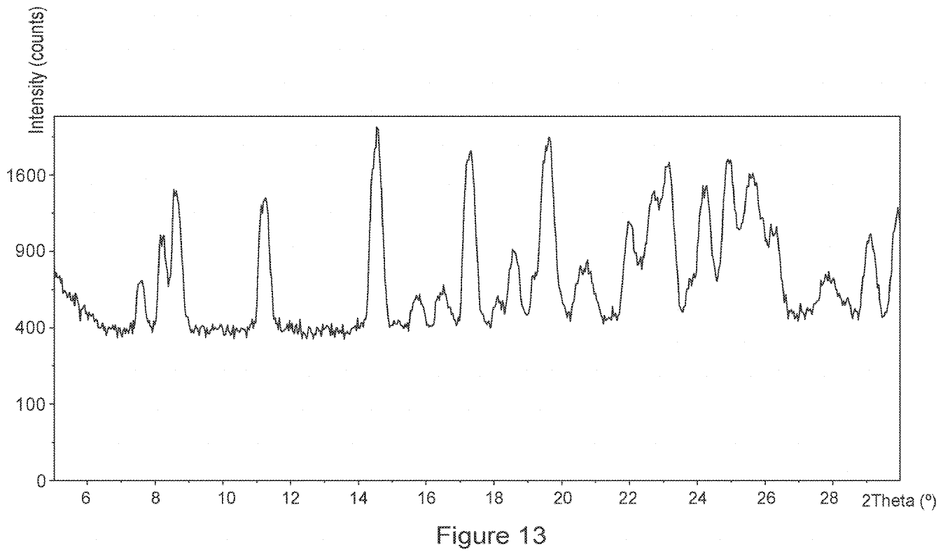

A crystalline form of Compound B and acetonitrile (“acetonitrile solvate”) can be characterized by an X-ray powder diffraction pattern, obtained as set forth in the Examples, having peaks at about 14.58, 17.36, 19.44, and 19.66±0.2° 2θ using Cu Kα radiation. The acetonitrile solvate optionally can be further characterized by an X-ray powder diffraction pattern having additional peaks at about 8.56, 11.29, 14.38, 17.16, 17.36, 19.44, 23.20, 24.83, and 25.60±0.2° 2θ using Cu Kα radiation. The acetonitrile solvate optionally can be further characterized by an X-ray powder diffraction pattern having additional peaks at about 11.10, 18.59, 20.79, 22.03, 22.66, 24.11, 24.31, 26.36, and 29.06 0.2° 2θ using Cu Kα radiation. The acetonitrile solvate optionally can be characterized by an X-ray powder diffraction pattern having peaks shown in Table 5 set forth in the Examples. In some embodiments, the acetonitrile solvate has an X-ray powder diffraction pattern substantially as shown in FIG. 13, wherein by “substantially” is meant that the reported peaks can vary by about ±0.2°.

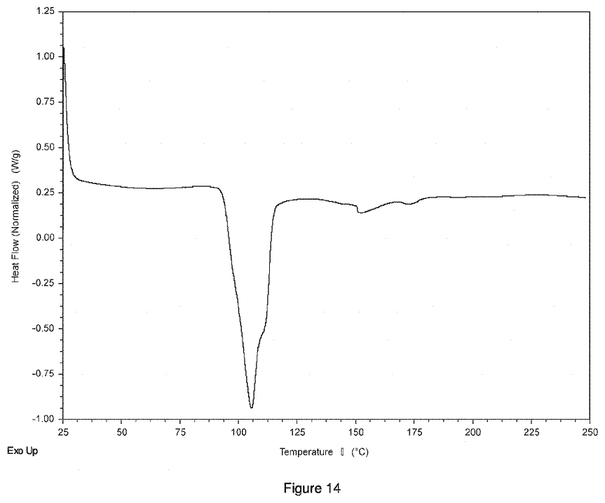

Differential scanning calorimetry (DSC) thermographs were obtained, as set forth in the Examples, for the acetonitrile solvate. The DSC curve indicates an endothermic transition at about 108° C.±3° C. Thus, in some embodiments, the acetonitrile solvate can be characterized by a DSC thermograph having a decomposition endotherm with an onset in a range of about 100° C. to about 115° C. For example, in some embodiments the acetonitrile solvate is characterized by DSC, as shown in FIG. 14.

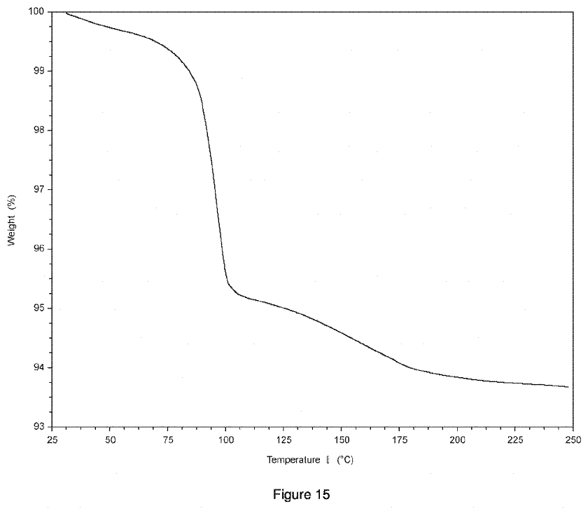

The acetonitrile solvate also can be characterized by thermogravimetric analysis (TGA). Thus, the acetonitrile solvate can be characterized by a weight loss in a range of about 5.5% to about 6.5% with an onset temperature in a range of about 65° C. to about 85° C. For example, the acetonitrile solvate can be characterized by a weight loss of about 6.5%, up to about 200° C. In some embodiments, the acetonitrile solvate has a thermogravimetric analysis substantially as depicted in FIG. 15, wherein by “substantially” is meant that the reported TGA features can vary by about ±5° C.

### Dichloroethane Solvate

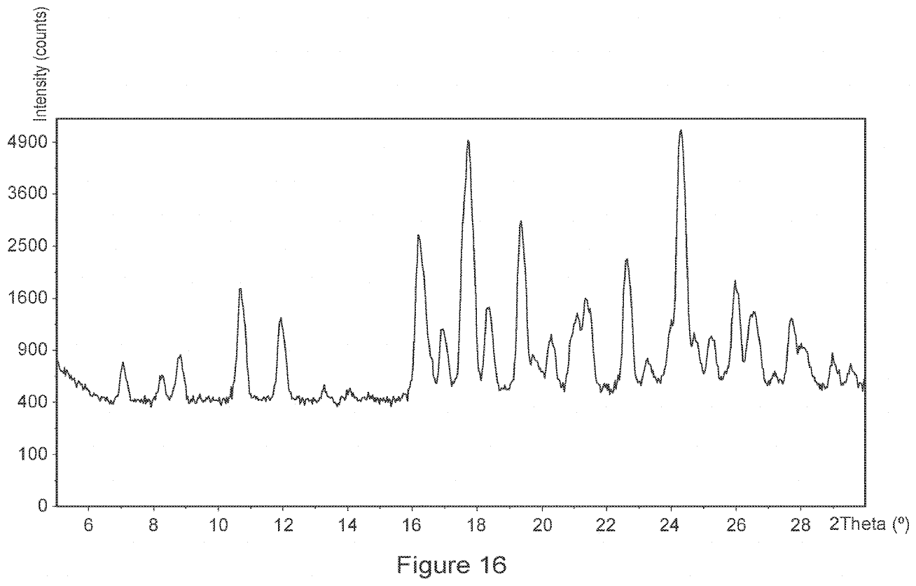

A crystalline form of Compound B and dichloroethane (“dichloroethane solvate”) can be characterized by an X-ray powder diffraction pattern, obtained as set forth in the Examples, having peaks at about 16.18, 17.54, 17.73, 19.33, and 24.26±0.2° 2θ using Cu Kα radiation. The dichloroethane solvate optionally can be further characterized by an X-ray powder diffraction pattern having additional peaks at about 10.67, 18.31, 21.35, 25.94, 26.43, and 26.59±0.2° 2θ using Cu Kα radiation. The dichloroethane solvate optionally can be further characterized by an X-ray powder diffraction pattern having additional peaks at about 11.91, 16.91, 20.26, 21.00, 21.51, 25.19, 27.68, and 28.13±0.2° 2θ using Cu Kα radiation. The dichloroethane solvate optionally can be characterized by an X-ray powder diffraction pattern having peaks shown in Table 6 set forth in the Examples. In some embodiments, the dichloroethane solvate has an X-ray powder diffraction pattern substantially as shown in FIG. 16, wherein by “substantially” is meant that the reported peaks can vary by about ±0.2°.

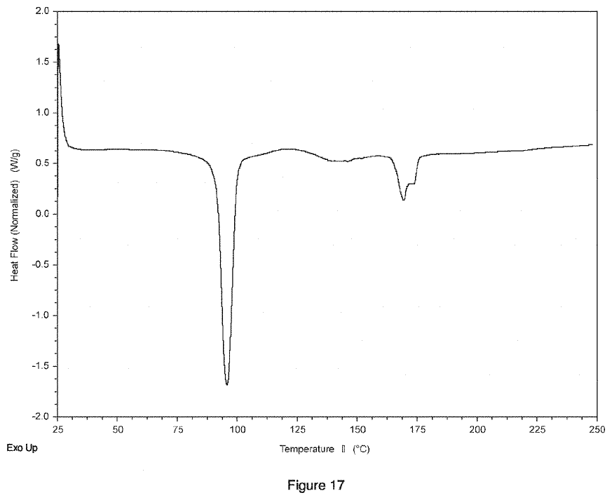

Differential scanning calorimetry (DSC) thermographs were obtained, as set forth in the Examples, for the dichloroethane solvate. The DSC curve indicates an endothermic transition at about 95° C.±3° C. Thus, in some embodiments, the dichloroethane solvate can be characterized by a DSC thermograph having a decomposition endotherm with an onset in a range of about 90° C. to about 100° C. For example, in some embodiments the dichloroethane solvate is characterized by DSC, as shown in FIG. 17.

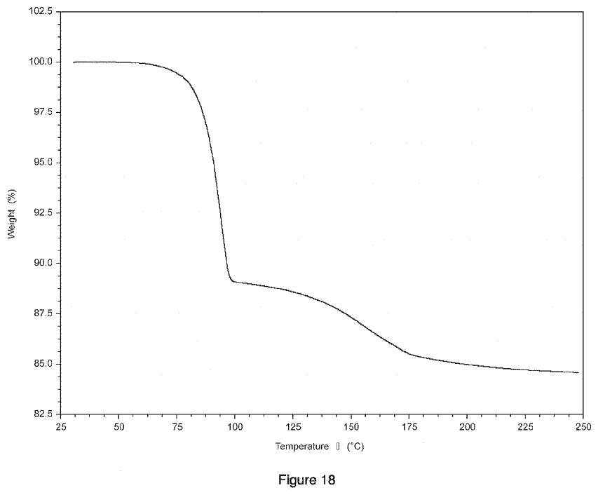

The dichloroethane solvate also can be characterized by thermogravimetric analysis (TGA). Thus, the dichloroethane solvate can be characterized by a weight loss in a range of about 14% to about 16% with an onset temperature in a range of about 70° C. to about 90° C. For example, the dichloroethane solvate can be characterized by a weight loss of about 15%, up to about 200° C. In some embodiments, the dichloroethane solvate has a thermogravimetric analysis substantially as depicted in FIG. 18, wherein by “substantially” is meant that the reported TGA features can vary by about ±5° C.

### Nitromethane Solvate

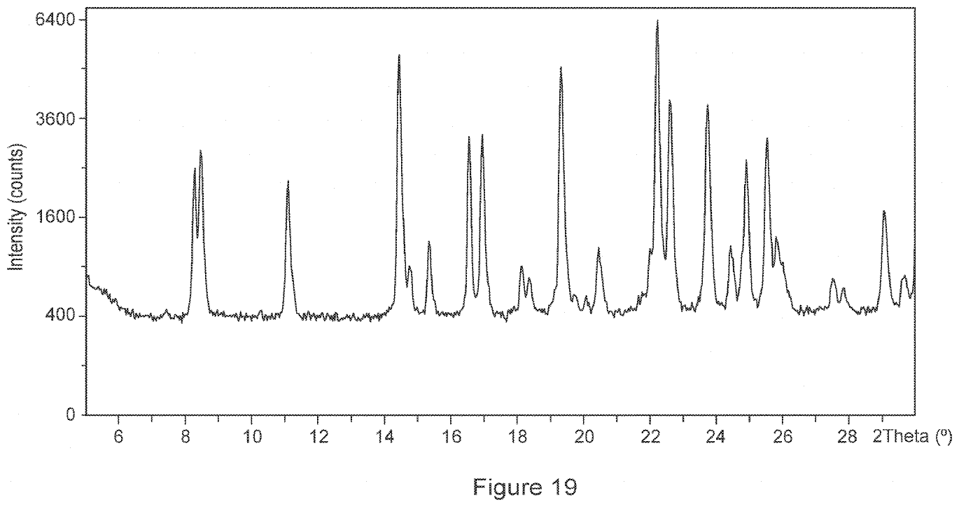

A crystalline form of Compound B and nitromethane (“nitromethane solvate”) can be characterized by an X-ray powder diffraction pattern, obtained as set forth in the Examples, having peaks at about 14.44, 19.32, 22.22, and 22.61±0.2° 2θ using Cu Kα radiation. The nitromethane solvate optionally can be further characterized by an X-ray powder diffraction pattern having additional peaks at about 8.27, 8.48, 16.55, 16.95, 23.74, and 25.53±0.2° 2θ using Cu Kα radiation. The nitromethane solvate optionally can be further characterized by an X-ray powder diffraction pattern having additional peaks at about 11.09, 15.35, 20.46, 24.44, 24.92, 25.92 and 29.07±0.2° 2θ using Cu Kα radiation. The nitromethane solvate optionally can be characterized by an X-ray powder diffraction pattern having peaks shown in Table 7 set forth in the Examples. In some embodiments, the nitromethane solvate has an X-ray powder diffraction pattern substantially as shown in FIG. 19, wherein by “substantially” is meant that the reported peaks can vary by about ±0.2°.

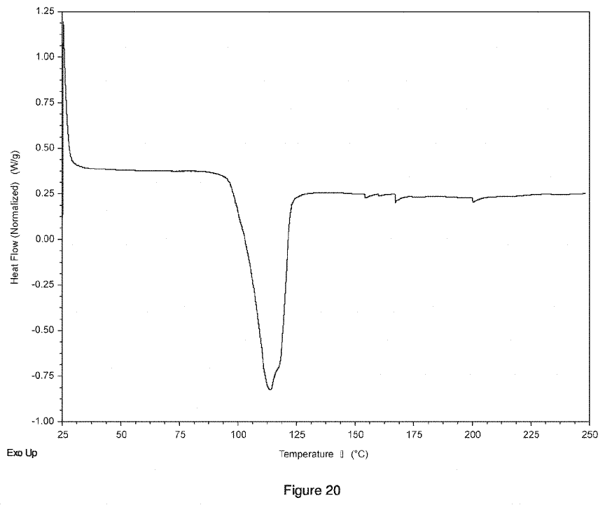

Differential scanning calorimetry (DSC) thermographs were obtained, as set forth in the Examples, for the nitromethane solvate. The DSC curve indicates an endothermic transition at about 112° C.±3° C. Thus, in some embodiments, the nitromethane solvate can be characterized by a DSC thermograph having a decomposition endotherm with an onset in a range of about 105° C. to about 120° C. For example, in some embodiments the nitromethane solvate is characterized by DSC, as shown in FIG. 20.

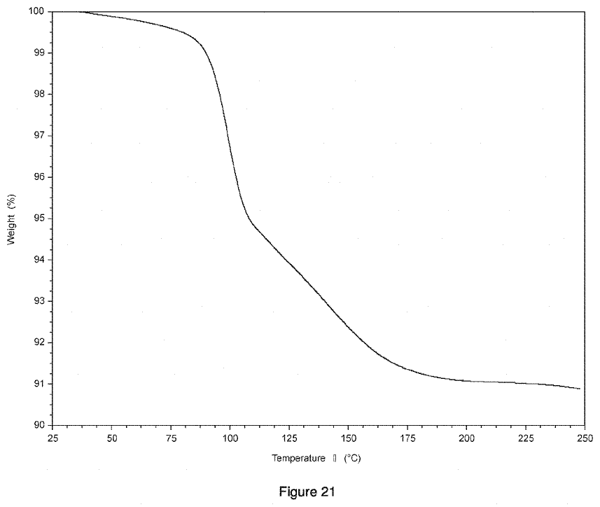

The nitromethane solvate also can be characterized by thermogravimetric analysis (TGA). Thus, the nitromethane solvate can be characterized by a weight loss in a range of about 7.9% to about 9.9% with an onset temperature in a range of about 75° C. to about 95° C. For example, the nitromethane solvate can be characterized by a weight loss of about 8.9%, up to about 200° C. In some embodiments, the nitromethane solvate has a thermogravimetric analysis substantially as depicted in FIG. 21, wherein by “substantially” is meant that the reported TGA features can vary by about ±5° C.

### PHARMACEUTICAL COMPOSITIONS

Also provided herein are pharmaceutical compositions comprising a form of Compound B or a salt thereof described herein; and a pharmaceutically acceptable carrier. In embodiments, the carrier can comprise an excipient.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The compositions described herein can be formulated for any form of administration. In various cases, the composition is for oral administration. In various cases, the composition is in tablet form.

The phrase “pharmaceutically acceptable carrier” as used herein means a pharmaceutically acceptable material, composition, or vehicle, such as a liquid or solid filler, diluent, excipient, solvent or encapsulating material. As used herein the language “pharmaceutically acceptable carrier” includes buffers, sterile water for injection, solvents, dispersion media, coatings, antibacterial and antifungal agents, isotonic and absorption delaying agents, and the like, compatible with pharmaceutical administration. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions provided herein are non-pyrogenic, i.e., do not induce significant temperature elevations when administered to a patient.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions as excipients.

Examples of pharmaceutically acceptable antioxidants as excipients include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

A pharmaceutical composition may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars and the like into the compositions. In addition, prolonged absorption of an injectable pharmaceutical form may be brought about by the inclusion of agents which delay absorption such as aluminum monostearate and gelatin.

In some cases, in order to prolong the effect of one or more compounds provided herein, it is desirable to slow the absorption of the compound from subcutaneous or intramuscular injection. For example, delayed absorption of a parenterally administered compound can be accomplished by dissolving or suspending the compound in an oil vehicle.

The composition should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the methods of preparation are freeze-drying (lyophilization), which yields a powder of the active ingredient plus any additional desired ingredient from a previously sterile-filtered solution thereof.

Injectable depot forms can be made by forming microencapsule or nanoencapsule matrices of a compound provided herein in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes, microemulsions or nanoemulsions, which are compatible with body tissue.

In some embodiments, the polymorphs and salts disclosed herein are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811, which is incorporated herein by reference in its entirety.

The pharmaceutical compositions can be included in a container, pack, or dispenser together with instructions for administration.

### METHODS OF USE

The forms of Compound B or a salt thereof disclosed herein, or the pharmaceutical compositions described herein, may be used in the treatment or prevention of heart failure, including but not limited to: acute (or decompensated) congestive heart failure, and chronic congestive heart failure; particularly diseases associated with systolic heart dysfunction.

Also provided herein are methods of treating or preventing heart failure in a subject in need thereof comprising administering to the subject one or more of the forms of Compound B or a salt thereof disclosed herein, or one or more of the pharmaceutical compositions described herein in an amount effective to treat or prevent heart failure. Further provided are methods for the use of the disclosed forms of Compound B, or compositions thereof, for the treatment or prevention of heart failure, including but not limited to: acute (or decompensated) congestive heart failure, and chronic congestive heart failure.

Also provided herein is the use of the forms of Compound B disclosed herein or a salt thereof, or the pharmaceutical compositions described herein, in the manufacture of a medicament for the treatment or prevention of heart failure. In some embodiments, the present disclosure provides use of the forms of Compound B or a salt thereof disclosed herein, or the pharmaceutical compositions described herein, in the manufacture of a medicament for the treatment of acute (or decompensated) congestive heart failure, and chronic congestive heart failure.

In some embodiments, the forms of Compound B or a salt thereof disclosed herein are used in the treatment or prevention of heart failure with reduced ejection fraction (HFrEF) or systolic heart failure, dilated cardiomyopathy, postpartum cardiomyopathy, idiopathic cardiomyopathy, pediatric HFrEF, chemotherapy-induced heart failure, heart failure associated with muscular dystrophy, bi-ventricular HFrEF, HFrEF with pulmonary hypertension, heart failure with preserved ejection fraction (HFpEF) with right ventricular dysfunction, pulmonary hypertension with right ventricular dysfunction, scleroderma with pulmonary hypertension, right ventricular dysfunction, Chagas disease, or myocarditis. In some embodiments, provided herein are methods of treating or preventing heart failure with reduced ejection fraction or systolic heart failure, dilated cardiomyopathy, postpartum cardiomyopathy, idiopathic cardiomyopathy, pediatric HFrEF, chemotherapy-induced heart failure, heart failure associated with muscular dystrophy, bi-ventricular HFrEF, HFrEF with pulmonary hypertension, heart failure with preserved ejection fraction (HFpEF) with right ventricular dysfunction, pulmonary hypertension with right ventricular dysfunction, scleroderma with pulmonary hypertension, right ventricular dysfunction, Chagas disease, or myocarditis, which methods comprise administering to a subject in need thereof an effective amount of one or more forms of Compound B or a salt thereof disclosed herein. Also provided herein is the use of one or more forms of Compound B or a salt thereof disclosed herein in the manufacture of a medicament for the treatment or prevention of heart failure with reduced ejection fraction or systolic heart failure, dilated cardiomyopathy, postpartum cardiomyopathy, idiopathic cardiomyopathy, pediatric HFrEF, chemotherapy-induced heart failure, heart failure associated with muscular dystrophy, bi-ventricular HFrEF, HFrEF with pulmonary hypertension, heart failure with preserved ejection fraction (HFpEF) with right ventricular dysfunction, pulmonary hypertension with right ventricular dysfunction, scleroderma with pulmonary hypertension, right ventricular dysfunction, Chagas disease, or myocarditis.

In some embodiments, the dilated cardiomyopathy is selected from the group consisting of genetic dilated cardiomyopathy, peripartum cardiomyopathy (e.g., post-partum cardiomyopathy), idiopathic dilated cardiomyopathy, post-infectious dilated cardiomyopathy, toxin-induced dilated cardiomyopathy, and nutritional deficiency dilated cardiomyopathy. In some embodiments, the pediatric HFrEF occurs in pediatric patients with univentricular hearts or a single ventricle or patients post Fontan or Fontan-Kreutzer procedure. In some embodiments, the pediatric HFrEF is pediatric heart failure associated with congenital heart disease. In some embodiments, the chemotherapy-induced heart failure is selected from the group consisting of chemotherapy-induced left ventricular dysfunction, radiation-induced heart failure, heart failure resulting from anthracycline treatment (including but not limited to doxorubicin, epirubicin, and daunorubicin), heart failure resulting from antiERBB2 treatment (including but not limited to trastuzumab and lapatinib), heart failure resulting from VEGF inhibitor treatment (including but not limited to bevacizumab), and heart failure resulting from tyrosine-kinase inhibitor treatment (including but not limited to imatinib, dasatinib, nilotinim, sorafenib, and sunitinib). In some embodiments, the heart failure associated with muscular dystrophy is selected from the group consisting of heart failure associated with Duchenne muscular dystrophy, heart failure associated with Becker muscular dystrophy, heart failure associated with myotonic dystrophy (e.g., Steinert's disease), heart failure associated with laminopathies such as Emery-Dreifuss muscular dystrophy (EDMD), including both X-linked EDMD and autosomal dominant EDMD, heart failure associated with facioscapulohumeral muscular dystrophy (FSHMD), heart failure associated with Limb-girdle muscular dystrophy, including sarcoglycanopathies and the autosomal dominant form of the disease, and heart failure associated with congenital muscular dystrophy. In some embodiments, the pulmonary hypertension with right ventricular dysfunction is associated with high left ventricular (diastolic) pressure in HFrEF or high left ventricular (diastolic) pressure in HFpEF.

“Treatment” or “treating” includes one or more of : a) inhibiting a disease or disorder; b) slowing or arresting the development of clinical symptoms of a disease or disorder; and/or c) relieving a disease or disorder that is, causing the regression of clinical symptoms. The term covers both complete and partial reduction of the condition or disorder, and complete or partial reduction of clinical symptoms of a disease or disorder. Thus, the forms of Compound B described herein, or the pharmaceutical compositions described herein may prevent an existing disease or disorder from worsening, assist in the management of the disease or disorder, or reduce or eliminate the disease or disorder. “Prevention,” that is, causing the clinical symptoms of the disease or disorder not to develop, includes the prophylactic administration of a pharmaceutical formulation described herein to a subject (i.e., an animal, preferably a mammal, most preferably a human) believed to be in need of preventative treatment, such as, for example, chronic heart failure.

### EXAMPLES

### Methods

### X-Ray Powder Diffraction (XRPD)

X-ray powder diffraction (XRPD) data were obtained using a PANalytical X'Pert PRO diffractometer. Samples were scanned at ambient temperature in continuous mode from 5-30 or 5-45 degrees (2θ) with step size of 0.0334 degrees at 45 kV and 40 mA with CuKα radiation (1.54 Å). The incident beam path was equipped with a 0.02 rad soller slit, 15 mm mask, 4 degrees fixed anti-scatter slit and a programmable divergence slit. The diffracted beam was equipped with a 0.02 rad soller slit, programmable anti-scatter slit and a 0.02 mm nickel filter. Samples were prepared on a low background sample holder and placed on a spinning stage with a rotation time of 2 s.

### Differential Scanning Calorimetry (DSC)

Differential scanning calorimetry (DSC) analysis was conducted on a TA Instruments Discovery Series calorimeter at 10 degrees C/min from 30 to 250 degrees C. in a crimped, aluminum pan under dry nitrogen at 50 ml/min.

### Thermal Gravimetric Analysis (TGA)

Thermal gravimetric analysis (TGA) was performed on a TA Instruments Discovery Series analyzer at 10 degrees C/min from ambient temperature to 250 degrees C. in a platinum pan under dry nitrogen at 25 ml/min.

### Moisture Sorption

Moisture sorption data was collected using a dynamic vapor sorption (DVS) analyzer. Hygroscopicity was evaluated from 0 to 95% RH in increments of 5 or 10% RH. Data for adsorption and desorption cycles were collected. Equilibrium criteria were set at 0.002% weight change in 5 minute with a maximum equilibration time of 120 minutes.

### Solubility

An excess of solid was added to water to produce a suspension and dispersed for at least 24 at room temperature. Suspensions were filtered. Filtrate was analyzed by ultra performance liquid chromatography-ultraviolet (“UPLC-UV”), and compared against a standard curve to determine the solution concentration of the crystal form. Solids were analyzed by XRPD to determine the crystal form.

### Solid Stability

Drug substance was stored at 25° C./60% RH, 40° C./75% RH, 40° C./ambient or 60° C./ambient conditions. Chemical stability was determined at each time point by dissolving the drug substance in 50% acetonitrile water for ultra performance liquid chromatography (“UPLC”) analysis. Physical stability was determined by analyzing the solid by XRPD, DSC and TGA.

Free base crystalline Form I: Form I was initially prepared by slurry of the acetonitrile solvate in water (10 mg/mL) during a solubility screen. Form I melts at ˜175° C. and is non-hygroscopic. Solubility of Form I in water is 0.009 mg/mL. Form I was physically and chemically stable for 5 weeks when stored at 25° C./60% RH, 40° C./75% RH, 40° C./ambient or 60° C./ambient conditions

The free base crystalline Form I was characterized by an XRPD pattern comprising peaks in Table 1.

TABLE 1

| Pos. | FWHM | d-spacing | Height | Rel. Int. |
|---|---|---|---|---|
| [°2Th.] | [°2Th.] | [Å] | [cts] | [%] |
| 8.31 | 0.16 | 10.65 | 392.30 | 9.59 |
| 10.20 | 0.19 | 8.67 | 449.13 | 10.98 |
| 12.16 | 0.19 | 7.28 | 175.14 | 4.28 |
| 13.11 | 0.19 | 6.75 | 816.40 | 19.96 |
| 14.07 | 0.19 | 6.30 | 3882.17 | 94.93 |
| 16.65 | 0.23 | 5.32 | 2764.80 | 67.61 |
| 18.34 | 0.16 | 4.84 | 584.69 | 14.30 |
| 19.36 | 0.26 | 4.58 | 703.97 | 17.21 |
| 19.84 | 0.16 | 4.47 | 773.35 | 18.91 |
| 20.42 | 0.19 | 4.35 | 4089.47 | 100.00 |
| 21.49 | 0.23 | 4.14 | 2640.84 | 64.58 |
| 22.21 | 0.16 | 4.00 | 591.68 | 14.47 |
| 22.57 | 0.19 | 3.94 | 902.96 | 22.08 |
| 23.39 | 0.19 | 3.80 | 1084.35 | 26.52 |
| 24.70 | 0.13 | 3.60 | 513.13 | 12.55 |
| 25.27 | 0.13 | 3.52 | 1612.02 | 39.42 |
| 25.60 | 0.19 | 3.48 | 2148.99 | 52.55 |
| 26.31 | 0.19 | 3.39 | 766.52 | 18.74 |
| 26.97 | 0.16 | 3.31 | 1092.48 | 26.71 |
| 28.02 | 0.13 | 3.18 | 1037.99 | 25.38 |
| 28.49 | 0.19 | 3.13 | 1572.64 | 38.46 |
| 28.91 | 0.13 | 3.09 | 632.12 | 15.46 |

Free base crystalline Form II: Form II was initially prepared by precipitation at ambient temperature after dissolving the acetonitrile solvate in 30% hydroxypropyl-β-cyclodextrin (20 mg/mL) in a formulation screen. The monohydrate converts to free base crystalline Form I when slurried in water.

The free base crystalline Form II was characterized by an XRPD pattern comprising peaks in Table 2.

TABLE 2

| Pos. | FWHM | d-spacing | Height | Rel. Int. |
|---|---|---|---|---|
| [°2Th.] | [°2Th.] | [Å] | [cts] | [%] |
| 6.19 | 0.13 | 14.28 | 375.71 | 14.31 |
| 9.96 | 0.10 | 8.88 | 296.29 | 11.29 |
| 12.37 | 0.13 | 7.15 | 556.33 | 21.19 |
| 15.40 | 0.19 | 5.75 | 1593.28 | 60.70 |
| 16.04 | 0.19 | 5.53 | 738.30 | 28.13 |
| 16.97 | 0.19 | 5.22 | 1067.93 | 40.68 |
| 17.65 | 0.13 | 5.02 | 1137.41 | 43.33 |
| 18.57 | 0.13 | 4.78 | 2625.01 | 100.00 |
| 19.32 | 0.10 | 4.59 | 1058.60 | 40.33 |
| 20.10 | 0.13 | 4.42 | 955.96 | 36.42 |
| 20.54 | 0.13 | 4.32 | 705.31 | 26.87 |
| 21.56 | 0.10 | 4.12 | 821.48 | 31.29 |
| 23.08 | 0.16 | 3.85 | 1387.95 | 52.87 |
| 23.44 | 0.10 | 3.80 | 974.51 | 37.12 |
| 23.83 | 0.16 | 3.73 | 1922.56 | 73.24 |
| 24.22 | 0.23 | 3.67 | 1375.22 | 52.39 |
| 24.95 | 0.16 | 3.57 | 748.97 | 28.53 |
| 25.51 | 0.23 | 3.49 | 676.61 | 25.78 |
| 25.99 | 0.19 | 3.43 | 879.21 | 33.49 |
| 26.42 | 0.16 | 3.37 | 852.45 | 32.47 |
| 26.76 | 0.16 | 3.33 | 784.06 | 29.87 |
| 27.51 | 0.19 | 3.24 | 1111.08 | 42.33 |
| 28.49 | 0.29 | 3.13 | 370.20 | 14.10 |
| 29.43 | 0.19 | 3.04 | 297.88 | 11.35 |

The XRPD peaks unique to each of the free base crystalline forms I-II disclosed herein are shown in Table 3.

TABLE 3

| Form | Some Peaks Unique to Each Form (KA1°) | | | | |
|---|---|---|---|---|---|
| Form I | 8.31 | 10.20 | 13.11 | 14.07 | 16.65 |
| Form II | 6.19 | 9.96 | 12.37 | 15.40 | 16.04 |

Hydrochloride Salt Form: Hydrochloride salt was initially prepared from slurry of Compound B in methyl tert-butyl ether (“MTBE”) with hydrochloric acid. The hydrochloride salt converts to free base crystalline Form I when slurried in water.

The hydrochloride salt form was characterized by an XRPD pattern comprising peaks in Table 4.

TABLE 4

| Pos. | FWHM | d-spacing | Height | Rel. Int. |
|---|---|---|---|---|
| [°2Th.] | [°2Th.] | [Å] | [cts] | [%] |
| 11.50 | 0.13 | 7.69 | 321.00 | 11.20 |
| 12.32 | 0.13 | 7.19 | 244.14 | 8.52 |
| 13.32 | 0.19 | 6.65 | 132.12 | 4.61 |
| 13.78 | 0.13 | 6.43 | 189.34 | 6.61 |
| 14.23 | 0.13 | 6.23 | 839.03 | 29.27 |
| 15.37 | 0.16 | 5.76 | 1555.76 | 54.28 |
| 17.54 | 0.13 | 5.06 | 324.17 | 11.31 |
| 17.83 | 0.10 | 4.97 | 679.65 | 23.71 |
| 18.13 | 0.10 | 4.89 | 1559.77 | 54.42 |
| 18.40 | 0.10 | 4.82 | 1075.35 | 37.52 |
| 18.68 | 0.10 | 4.75 | 741.85 | 25.88 |
| 18.94 | 0.10 | 4.69 | 686.01 | 23.93 |
| 19.07 | 0.10 | 4.65 | 720.54 | 25.14 |
| 19.73 | 0.10 | 4.50 | 408.47 | 14.25 |
| 20.00 | 0.13 | 4.44 | 2866.24 | 100.00 |
| 20.71 | 0.13 | 4.29 | 369.45 | 12.89 |
| 21.41 | 0.13 | 4.15 | 268.37 | 9.36 |
| 22.23 | 0.10 | 4.00 | 641.14 | 22.37 |
| 22.45 | 0.13 | 3.96 | 1370.84 | 47.83 |
| 22.62 | 0.10 | 3.93 | 1126.32 | 39.30 |
| 23.09 | 0.13 | 3.85 | 415.02 | 14.48 |
| 23.39 | 0.13 | 3.80 | 994.61 | 34.70 |
| 23.94 | 0.13 | 3.72 | 1171.84 | 40.88 |
| 24.42 | 0.13 | 3.65 | 736.86 | 25.71 |
| 24.84 | 0.16 | 3.58 | 1792.10 | 62.52 |
| 25.42 | 0.16 | 3.50 | 592.22 | 20.66 |
| 26.91 | 0.16 | 3.31 | 1474.55 | 51.45 |
| 27.39 | 0.13 | 3.26 | 736.65 | 25.70 |
| 27.71 | 0.13 | 3.22 | 1527.08 | 53.28 |
| 28.31 | 0.19 | 3.15 | 828.20 | 28.90 |
| 29.08 | 0.13 | 3.07 | 1115.69 | 38.93 |
| 29.38 | 0.13 | 3.04 | 533.37 | 18.61 |
| 29.80 | 0.16 | 3.00 | 418.94 | 14.62 |
| 30.58 | 0.13 | 2.92 | 328.94 | 11.48 |
| 31.38 | 0.13 | 2.85 | 399.71 | 13.95 |
| 32.25 | 0.19 | 2.78 | 197.63 | 6.90 |
| 32.71 | 0.19 | 2.74 | 255.49 | 8.91 |
| 33.65 | 0.16 | 2.66 | 262.46 | 9.16 |
| 34.09 | 0.16 | 2.63 | 470.11 | 16.40 |
| 35.98 | 0.16 | 2.50 | 352.25 | 12.29 |
| 38.09 | 0.16 | 2.36 | 565.60 | 19.73 |
| 38.68 | 0.19 | 2.33 | 296.46 | 10.34 |
| 40.01 | 0.19 | 2.25 | 593.91 | 20.72 |
| 40.59 | 0.19 | 2.22 | 294.28 | 10.27 |
| 41.30 | 0.13 | 2.19 | 300.34 | 10.48 |
| 42.09 | 0.16 | 2.15 | 676.35 | 23.60 |
| 43.04 | 0.26 | 2.10 | 279.69 | 9.76 |
| 44.39 | 0.24 | 2.04 | 466.51 | 16.28 |

Acetonitrile Solvate: The acetonitrile solvate was prepared during synthesis of Compound B. Final step of the synthesis was reverse phase purification in 25-70% acetonitrile/water with trifluoroacetic acid.

The acetonitrile solvate was characterized by an XRPD pattern comprising peaks in Table 5.

TABLE 5

| Pos. | FWHM | d-spacing | Height | Rel. Int. |
|---|---|---|---|---|
| [°2Th.] | [°2Th.] | [Å] | [cts] | [%] |
| 7.51 | 0.23 | 11.78 | 288.31 | 17.06 |
| 8.17 | 0.16 | 10.83 | 624.34 | 36.95 |
| 8.56 | 0.13 | 10.33 | 1037.77 | 61.42 |
| 11.10 | 0.13 | 7.97 | 783.10 | 46.35 |
| 11.29 | 0.16 | 7.84 | 933.34 | 55.24 |
| 14.38 | 0.13 | 6.16 | 1145.30 | 67.79 |
| 14.58 | 0.19 | 6.08 | 1689.50 | 100.00 |
| 15.72 | 0.32 | 5.64 | 192.37 | 11.39 |
| 16.43 | 0.32 | 5.39 | 213.76 | 12.65 |
| 17.16 | 0.16 | 5.17 | 1176.87 | 69.66 |
| 17.36 | 0.16 | 5.11 | 1339.88 | 79.31 |
| 18.59 | 0.32 | 4.77 | 518.44 | 30.69 |
| 19.44 | 0.16 | 4.57 | 1306.93 | 77.36 |
| 19.66 | 0.19 | 4.51 | 1562.01 | 92.45 |
| 20.79 | 0.52 | 4.27 | 402.27 | 23.81 |
| 22.03 | 0.29 | 4.04 | 747.22 | 44.23 |
| 22.66 | 0.19 | 3.92 | 1012.15 | 59.91 |
| 23.20 | 0.26 | 3.83 | 1238.10 | 73.28 |
| 24.11 | 0.16 | 3.69 | 913.83 | 54.09 |
| 24.31 | 0.13 | 3.66 | 1000.77 | 59.24 |
| 24.83 | 0.29 | 3.59 | 1223.72 | 72.43 |
| 25.60 | 0.32 | 3.48 | 1220.48 | 72.24 |
| 26.36 | 0.10 | 3.38 | 658.10 | 38.95 |
| 27.76 | 0.45 | 3.21 | 318.09 | 18.83 |
| 29.06 | 0.36 | 3.07 | 618.54 | 36.61 |

Dichloroethane Solvate: The dichloroethane solvate was initially prepared by precipitation with 1 volume of water from dichloroethane/toluene 1:1 or dichloroethane/heptane 1:1 (10 mg/mL) during a high-throughput polymorph screen.

The dichloroethane solvate was characterized by an XRPD pattern comprising peaks in Table 6.

TABLE 6

| Pos. | FWHM | d-spacing | Height | Rel. Int. |
|---|---|---|---|---|
| [°2Th.] | [°2Th.] | [Å] | [cts] | [%] |
| 7.04 | 0.13 | 12.56 | 315.52 | 6.81 |
| 8.26 | 0.19 | 10.71 | 210.75 | 4.55 |
| 8.80 | 0.23 | 10.04 | 409.05 | 8.83 |
| 10.67 | 0.19 | 8.29 | 1325.15 | 28.61 |
| 11.91 | 0.23 | 7.43 | 854.44 | 18.45 |
| 13.28 | 0.19 | 6.67 | 98.56 | 2.13 |
| 16.18 | 0.19 | 5.48 | 2218.70 | 47.90 |
| 16.91 | 0.26 | 5.24 | 749.33 | 16.18 |
| 17.54 | 0.13 | 5.06 | 2374.77 | 51.27 |
| 17.73 | 0.19 | 5.00 | 4529.82 | 97.80 |
| 18.31 | 0.23 | 4.84 | 1040.54 | 22.46 |
| 19.33 | 0.19 | 4.59 | 2483.39 | 53.62 |
| 20.26 | 0.23 | 4.38 | 595.61 | 12.86 |
| 21.00 | 0.19 | 4.23 | 787.11 | 16.99 |
| 21.35 | 0.10 | 4.16 | 1171.96 | 25.30 |
| 21.51 | 0.10 | 4.13 | 925.35 | 19.98 |
| 22.59 | 0.19 | 3.94 | 1760.09 | 38.00 |
| 23.23 | 0.19 | 3.83 | 372.03 | 8.03 |
| 24.26 | 0.26 | 3.67 | 4631.85 | 100.00 |
| 25.19 | 0.13 | 3.54 | 624.66 | 13.49 |
| 25.94 | 0.16 | 3.43 | 1329.98 | 28.71 |
| 26.43 | 0.13 | 3.37 | 903.12 | 19.50 |
| 26.59 | 0.13 | 3.35 | 951.53 | 20.54 |
| 27.68 | 0.19 | 3.22 | 856.41 | 18.49 |
| 28.13 | 0.19 | 3.17 | 497.45 | 10.74 |
| 28.97 | 0.13 | 3.08 | 417.84 | 9.02 |

Nitromethane Solvate: The nitromethane solvate was prepared by evaporation at ambient temperature from nitromethane (10 mg/mL) during a high-throughput polymorph screen.

The nitromethane solvate was characterized by an XRPD pattern comprising peaks in Table 7.

TABLE 7

| Pos. | FWHM | d-spacing | Height | Rel. Int. |
|---|---|---|---|---|
| [°2Th.] | [°2Th.] | [Å] | [cts] | [%] |
| 8.27 | 0.10 | 10.70 | 1964.09 | 33.74 |
| 8.48 | 0.10 | 10.43 | 2388.37 | 41.03 |
| 11.09 | 0.13 | 7.98 | 1798.21 | 30.89 |
| 14.44 | 0.13 | 6.13 | 4654.03 | 79.96 |
| 15.35 | 0.13 | 5.77 | 844.36 | 14.51 |
| 16.55 | 0.10 | 5.36 | 2659.09 | 45.68 |
| 16.95 | 0.10 | 5.23 | 2682.64 | 46.09 |
| 18.14 | 0.10 | 4.89 | 530.21 | 9.11 |
| 19.32 | 0.13 | 4.59 | 4353.55 | 74.80 |
| 20.46 | 0.13 | 4.34 | 746.39 | 12.82 |
| 22.22 | 0.10 | 4.00 | 5820.60 | 100.00 |
| 22.61 | 0.13 | 3.93 | 3641.67 | 62.57 |
| 23.74 | 0.16 | 3.75 | 3512.89 | 60.35 |
| 24.44 | 0.13 | 3.64 | 721.08 | 12.39 |
| 24.92 | 0.13 | 3.57 | 2149.84 | 36.94 |
| 25.53 | 0.13 | 3.49 | 2695.33 | 46.31 |
| 25.92 | 0.13 | 3.44 | 627.66 | 10.78 |
| 27.51 | 0.13 | 3.24 | 359.73 | 6.18 |
| 27.83 | 0.10 | 3.21 | 270.39 | 4.65 |
| 29.07 | 0.13 | 3.07 | 1309.55 | 22.50 |
| 29.63 | 0.16 | 3.01 | 376.89 | 6.48 |

What is claimed:

1. A free base anhydrous crystalline form of Compound B (“Form I”), characterized by an X-ray powder diffraction (XRPD) pattern comprising peaks at 8.31, 10.20, 13.11, 14.07, and 16.65±0.2° 2θ using Cu Kα radiation.

2. The crystalline form of claim 1, further characterized by XRPD pattern peaks at 20.42, 21.49, 22.57, 23.39, 25.27, and 25.60±0.2° 2θ using Cu Kα radiation.

3. The crystalline form of claim 2, further characterized by XRPD pattern peaks at 18.34, 19.36, 19.84, 22.21, 24.70, 26.31, 26.97, 28.02, 28.49, and 28.91±0.2° 2θ using Cu Kα radiation.

4. The crystalline form of any one of claims 1 to 3, having an XRPD pattern substantially as shown in FIG. 1.

5. The crystalline form of any one of claims 1 to 4, having an endothermic transition at 170° C. to 180° C., as measured by differential scanning calorimetry.

6. The crystalline form of claim 5, wherein the endothermic transition is at 175° C.±3° C.

7. The crystalline form of any one of claims 1 to 6, having a dynamic vapor sorption (“DVS”) substantially as shown in FIG. 4.

8. The crystalline form of any one of claims 1 to 7, having a thermogravimetric analysis (“TGA”) substantially as shown in FIG. 3.

9. A free base monohydrate crystalline form of Compound B (“Form II”), characterized by an X-ray powder diffraction (XRPD) pattern comprising peaks at 6.19, 9.96, 12.37, 15.40, and 16.04±0.2° 2θ using Cu Kα radiation.

10. The crystalline form of claim 9, further characterized by XRPD pattern peaks at 16.97, 17.65, 18.57, 19.32, 20.10, 21.56, 23.08, 23.44, 23.83, 24.22, and 27.51±0.2° 2θ using Cu Kα radiation.

11. The crystalline form of claim 10, further characterized by XRPD pattern peaks at 20.54, 24.95, 25.51, 26.76, 28.49, and 29.43±0.2° 2θ using Cu Kα radiation.

12. The crystalline form of any one of claims 9 to 11, having an XRPD pattern substantially as shown in FIG. 5.

13. The crystalline form of any one of claims 9 to 12, having an endothermic transition at 100° C. to 115° C., as measured by differential scanning calorimetry.

14. The crystalline form of claim 13, wherein the endothermic transition is at 106° C.±3° C.

15. The crystalline form of any one of claims 9 to 14, having a dynamic vapor sorption (“DVS”) substantially as shown in FIG. 8.

16. The crystalline form of any one of claims 9 to 15, having a thermogravimetric analysis (“TGA”) substantially as shown in FIG. 7.

17. A crystalline form of Compound B hydrochloride salt, characterized by an X-ray powder diffraction (XRPD) pattern comprising peaks at 15.37, 18.13, 20.00, 22.45, 24.84, 26.91, and 27.71±0.2° 2θ using Cu Kα radiation.

18. The crystalline form of claim 17, further characterized by XRPD pattern peaks at 14.23, 17.83, 18.40, 18.68, 18.94, 19.07, 22.23, 22.45, 22.62, 23.39, 23.94, 24.42, 25.42, 27.39, 28.31, 29.08, 40.01, and 42.09±0.2° 2θ using Cu Kα radiation.

19. The crystalline form of claim 18, further characterized by XRPD pattern peaks at 11.50, 17.54, 19.73, 20.71, 23.09, 29.38, 29.80, 31.38, 34.09, 38.09, and 44.39±0.2° 2θ using Cu Kα radiation.

20. The crystalline form of any one of claims 17 to 19, having an XRPD pattern substantially as shown in FIG. 10.

21. The crystalline form of any one of claims 17 to 20, having an endothermic transition at 140° C. to 155° C., as measured by differential scanning calorimetry.

22. The crystalline form of claim 21, wherein the endothermic transition is at 148° C.±3° C.

23. The crystalline form of any one of claims 17 to 22, having a thermogravimetric analysis (“TGA”) substantially as shown in FIG. 12.

24. A crystalline form of Compound B and acetonitrile, characterized by an X-ray powder diffraction (XRPD) pattern comprising peaks at 14.58, 17.36, 19.44, and 19.66±0.2° 2θ using Cu Kα radiation.

25. The crystalline form of claim 24, further characterized by XRPD pattern peaks at 8.56,

11. 29, 14.38, 17.16, 17.36, 19.44, 23.20, 24.83, and 25.60±0.2° 2θ using Cu Kα radiation.

26. The crystalline form of claim 25, further characterized by XRPD pattern peaks at 11.10,

18. 59, 20.79, 22.03, 22.66, 24.11, 24.31, 26.36, and 29.06±0.2° 2θ using Cu Kα radiation.

27. The crystalline form of any one of claims 24 to 26, having an XRPD pattern substantially as shown in FIG. 13.

28. The crystalline form of any one of claims 24 to 27, having an endothermic transition at 100° C. to 115 as measured by differential scanning calorimetry.

29. The crystalline form of claim 28, wherein the endothermic transition is at 108° C.±3° C.

30. The crystalline form of any one of claims 24 to 29, having a thermogravimetric analysis (“TGA”) substantially as shown in FIG. 15.

31. A crystalline form of Compound B and dichloroethane, characterized by an X-ray powder diffraction (XRPD) pattern comprising peaks at 16.18, 17.54, 17.73, 19.33, and 24.26±0.2° 2θ using Cu Kα radiation.

32. The crystalline form of claim 31, further characterized by XRPD pattern peaks at 10.67, 18.31, 21.35, 25.94, 26.43, and 26.59±0.2° 2θ using Cu Kα radiation.

33. The crystalline form of claim 32, further characterized by XRPD pattern peaks at 11.91, 16.91, 20.26, 21.00, 21.51, 25.19, 27.68, and 28.13 ±0.2° 2θ using Cu Kα radiation.

34. The crystalline form of any one of claims 31 to 33, having an XRPD pattern substantially as shown in FIG. 16.

35. The crystalline form of any one of claims 31 to 34, having an endothermic transition at 90° C. to 100° C., as measured by differential scanning calorimetry.

36. The crystalline form of claim 35, wherein the endothermic transition is at 95° C.±3° C.

37. The crystalline form of any one of claims 31 to 36, having a thermogravimetric analysis (“TGA”) substantially as shown in FIG. 18.

38. A crystalline form of Compound B and nitromethane, characterized by an X-ray powder diffraction (XRPD) pattern comprising peaks at 14.44, 19.32, 22.22, and 22.61±0.2° 2θ using Cu Kα radiation.

39. The crystalline form of claim 38, further characterized by XRPD pattern peaks at 8.27, 8.48, 16.55, 16.95, 23.74, and 25.53±0.2° 2θ using Cu Kα radiation.

40. The crystalline form of claim 39, further characterized by XRPD pattern peaks at 11.09, 15.35, 20.46, 24.44, 24.92, 25.92 and 29.07±0.2° 2θ using Cu Kα radiation.

41. The crystalline form of any one of claims 38 to 40, having an XRPD pattern substantially as shown in FIG. 19.

42. The crystalline form of any one of claims 38 to 41, having an endothermic transition at 105° C. to 120° C., as measured by differential scanning calorimetry.

43. The crystalline form of claim 42, wherein the endothermic transition is at 112° C.±3° C.

44. The crystalline form of any one of claims 38 to 43, having a thermogravimetric analysis (“TGA”) substantially as shown in FIG. 21.

45. A pharmaceutical composition comprising the crystalline form of Compound B or a salt thereof of any one of claims 1 to 44 and a pharmaceutically acceptable carrier.

46. A method of treating heart failure in a subject in need thereof comprising administering to the subject the crystalline form of Compound B or a salt thereof of any one of claims 1 to 44 or the composition of claim 45 in an amount effective to treat heart failure.
